(19) United States
(12) Patent Application Publication
(10) Pub. No.: US 2004/0058997 A1
(43) Pub. Date: Mar. 25, 2004

(54) Method for treatment of disorders of personal attachment and deficient social interaction

(76) Inventor: David Gordon Daniel, McLean, VA (US)

Correspondence Address: David Gordon Daniel, MD, PO Box 6207, McLean, VA, 22106, US

(21) Appl. No.: 10/252,716

(22) Filed: Sep. 24, 2002

(51) Int. Cl. A61K 31/19

(52) U.S. Cl. 514/557

(57) ABSTRACT

The present invention provides a process for treatment of central nervous system disorders characterized by interpersonal discomfort and awkwardness, diminished social approach and initiative, and paucity of interpersonal attachments and social interactions. Abnormal perceptions of interpersonal communication and peculiarities of social behavior commonly accompany these symptoms

Inhibited initiation of social behavior and personal attachment are cardinal symptoms of schizotypal personality disorder, schizoid personality disorder, paranoid personality disorder, avoidant personality disorder; pervasive developmental disorder, and Aspergerger's syndrome. These symptoms may also in the form of clinically significant social introversion that does not meet the threshold for a formal psychiatric disorder by current diagnostic standards such as DSM-IV.

The present treatment provides a process of symptomatic relief and stabilization of the course of these disorders.

### BACKGROUND OF THE INVENTION

A confluence of literature suggests that dopaminergic modulation plays a critical role in initiation and maintenance of normal social behavior and attachment in animals (Grierson et al, 1988; Melis and Argiolas, 1995;Grant et al, 1998; Stein, 1998; Mathew et al, 2001; and humans (Tihihonen et al, 1997; Breier et al, 1998; Laakso et al, 2000; Schneier et al, 2000; Condren et al, 2002). This literature suggests that in animals and humans derangements of normal homeostasis or modulation of central dopaminergic activity result in reductions of normal initiation and maintenance of social behaviors, social withdrawal, and loss of normal social attachment behavior.

In addition to social withdrawal per se imbalances of central dopaminergic activity may result in peculiarities of social behavior that elicit social rejection, social discomfort and other negative feedback from peers in response to social initiatives, thus reinforcing social withdrawal.

Current mechanistic explanations for the therapeutic effects of anticonvulsant drugs on epilepsy frequently cite voltage and use dependent block of voltage dependent sodium channels. In addition most anticonvulsant agents modulate activity of the excitatory neurotransmitter glutamate or facilitate the inhibitory neurotransmitter gamma amino butyric acid (GABA) or both (White, 1999). Glutaminergic and GABAergic neurons in the central nervous system synapse on and modulate the activity of dopaminergic neurons in both the cortex and subcortex (Barros and Leite, 1986; Sandoval and Palermo-neto, 1995; Agmo et al, 1997; Egan and Hyde, 2000). As an apparent downstream result of their effects on central GABA and glutamate activity various anticonvulsants have demonstrated modulatory effects on dopaminergic activity (Biggs et al, 1992; Okada et al, 1995; Okada et al, 1997; Southam et al, 1998; Eckerman et al, 2001; Yatham et al, 2002). These modulatory effects of anticonvulsants on dopaminergic activity have been demonstrated in brain areas felt relevant to social approach including the hippocampus (Murakami, 2001) and cortex (Ichikawa and Meltzer, 1999). Certain anticonvulsants appear to enhance dopamine activity in the cortex where dopaminergic deficiencies may inhibit social behavior and dampen it in the hippocampus where dopaminergic excess may further disorder social behavior.

Thus, by their modulation of dopamine activity, anticonvulsants should have the potential to treat disorders of social initiative and attachment as well as related behavioral peculiarities that may elicit negative social feedback.

Advantages of Anticonvulsants Over Other Agents that Alter Dopaminergic Function:

Anticonvulsants have generally favorable safety and tolerability profiles. Dopamine precursors such as 1-dopa and carbidopa/1-dopa elevate dopamine in an unregulated manner not infrequently causing psychosis, hypotension and nausea. Conventional neuroleptic agents such as haloperidol, chlorpromazine and others may cause sudden death, derangements in the hypothalamic-pituitary access or serious acute motor side effects such as dystonia, akathisia or parkinsonism or long-term debilitating motor disorders such as tardive dyskinesia. Newer generation a typical antipsychotic agents such as clozapine, risperidone, olanzapine, and aripiprazole while offering certain tolerability advantages over conventional neuroleptics in respect to iatrogenic movement disorders may be associated with bone marrow suppression, induction of seizures, effects on cardiac function, derangements in glucose lipid metabolism and in the hypothalamic-pituitary axis, and occasional serious acute motor side effects such as dystonia, akathisia or parkinsonism.

Stimulants such as amphetamine methylphenidate carry the risk of substance abuse, addiction, and induction of psychosis or mania.

Amantadine carries the risk of induction of psychosis.

Dopamine precursors, agonists, and reuptake blockers such as carbidopa/1-dopa, bromocriptine, and pramipexole have characteristic side effects involving the cardiovascular system, GI tract, motor system, may induce psychosis.

Benzodiazepines such as diazepam, lorazepam, clonazepam carry the risk of substance abuse, addiction, and dangerously disinhibited behavior.

Discussion of Previously Approved Patents:

U.S. Pat. No. 6,372,792 (Method for treating anxiety, anxiety disorders and insomnia) describes use of gabapentin in the treatment of anxiety disorders and sleep disturbances. In contrast to U.S. Pat. No. 6,372,792 the current invention does not purport to treat anxiety and does not rely upon sedation as its mechanism of action.

U.S. Pat. No. 6,342,515 (Remedy for neurodegenerative diseases) describes use of zonisamide to treat neurodegenerative diseases that present primarily with physical disorders of motor function. In contrast the current invention addresses disorders of behavior that are not considered neurodegenerative and do not have significant motor components.

### SUMMARY

The object is to provide a treatment for central nervous system disorders characterized by paucity, deficiencies and inhibition of social interaction. These disorders are extremely resistant to currently available interventions. The process described in this application utilizes adequate dosages of anticonvulsant treatments that directly or indirectly modulate activity of the neurotransmitter dopamine. The proposed treatment has advantages over other modulators of dopamine in that it lacks both the significant drug abuse potential and side effects characteristic of drugs that augment dopaminergic activity; and lacks the side effects characteristic of drugs that deplete dopamine or directly antagonize its activity at dopamine receptors.

### DESCRIPTION OF THE INVENTION

1. A invention is a process for treatment of central nervous system disorders characterized by any one or combination of the following: a) deficits in initiation and maintenance of personal attachment, social approach or interpersonal interaction; and/or b) subjective and objective awkwardness and discomfort in interpersonal interactions; c) and/or oddities or peculiarities of interpersonal communication and social behavior; and/ or d) subtle peculiarities and disorganization of thinking insufficient to warrant a specific DSM-IV axis I diagnosis.

2. The process comprises administration of an effective amount of an anticonvulsant that possesses the property of directly, indirectly, or by downstream mechanisms modulating the activity of the neurotransmitter dopamine within the brain. Since the modulatory effect of anticonvulsants on central dopaminergic activity is not critically bound to their anticonvulsant effects, the dosages required to treat the disorders described in this document may be higher, lower or the same than those required to treat epilepsy.

3. Examples of disorders that may benefit from the treatment process include schizoid personality disorder, schizotypal personality disorder, avoidant personality disorder, paranoid personality disorder, Aspberger's syndrome, pervasive developmental disorder, and clinically significant social introversion that does not meet the threshold for a formal psychiatric disorder by current diagnostic standards such as DSM-IV.

Anticonvulsant medications that may be effective in this process may be used for any period of time at maintenance doses that may be similar to or differ from those used to treat epilepsy. Examples of such medications and dosages include but are not limited to: 1) an adequate oral dosage of valproic acid, valproate, or its enteric coated derivatives; with a daily maintenance dosage, for example in the range of 15-60 mg/kg/d for adults and children; 2) an adequate oral dosage of carbamazepine with a daily maintenance dosage, for example, in the range of 820 mg/kg/d for adults and 10-35 mg/kg/d for children; 3) an adequate oral dosage of oxcarbazepine with a daily maintenance dosage, for example, in the range of 1200-2400 mg/d for adults and 20-50 mg/kg/d for children; 4) an adequate oral dosage of zonisamide with the daily maintenance dosage, for example, in the range of 200-600 mg/d for adults and 4-8 mg/kg/d for children; 5) an adequate oral dosage of lamotrigine with the daily dosage, for example, in the range of 100 to 500 mg/day for adults and 1-15 mg/kg/d for children; 6) an adequate oral dosage of ethosuximide with the daily maintenance dosage, for example, in the range of 500-1500 mg/d of body weight for adults and 10-40 mg/kg/d for children; 7) an adequate oral dosage of felbamate with the daily maintenance dosage, for example in the range of 1200-3600 mg/day in adults or 15-45 mg/kg/day for children; 8) the treatment is an adequate oral dosage of levetiracetam; including for example a daily maintenance dosage in the range of 1200-3600 mg/d for adults and 15-45 mg/kg/day for children; 9) the treatment is an adequate oral dosage of methsuximide; including for example, a daily maintenance dosage in the range of 300-1200 mg/d for adults and 150-1200 mg/d for children; 10) the treatment is an adequate oral dosage of phenytoin; and the daily maintenance dosage for example is in the range of 4-7 mg/kg for adults and 5-10 mg/kg/d for children; 11) the treatment is an adequate oral dosage of primidone; and the daily maintenance dosage for example is in the range of 750-2000 mg/d for adults and 1-25 mg/kg/d for children; 12) the treatment is an adequate oral dosage of tigabine, including for example, a daily maintenance dosage in the range of 32-56 mg/d for adults and 4-32 mg/d for children; 13) the treatment is an adequate oral dosage of topiramate; including for example a daily maintenance dosage in the range of 200-600 mg/d for adults and 5-9 mg/kg/d for children.

### REFERENCES

Agmo A, Medrano A, Garrido N, Alonso P. GABAergic drugs inhibit amphetamine-induced distractibility in the rat. Pharmacol Biochem Behav 1997 September;58(1):110-26

Barros H M, Leite J R. Effects of acute and chronic carbamazepine administration on apomorphine-elicited stereotypy. Eur J Pharmacol Apr. 29, 1986;123(3):345-9.

Biggs C S, Pearce B R, Fowler L J, Whitton P S. Regional effects of sodium valproate on extracellular concentrations of 5-hydroxytryptamine, dopamine, and their metabolites in the rat brain: an in vivo microdialysis study.

Breier A, Kestler L, Adler C, Elman I, Wiesenfeld N, Malhotra A, Pickar D. Dopamine D2 Receptor Density and Personal Detachment in Healthy Subjects. Am J Psychiatry October 1998;155:1440-42

Condren R M, Sharifi N, Thakore J H. A preliminary study of dopamine-mediated prolactin inhibition in generalized social phobia. Psychiatry Res Aug. 5, 2002;111(1):8792.

Egan M, Hyde T. “Schizophrenia: Neurobiology” in Kaplan & Sadock's comprehensive textbook of psychiatry/VII, Philadelphia, 2002.

Eckermann K, Beasley A, Yang P, Gaytan O, Swann A, Dafny N. Methylphenidate sensitization is modulated by valproate. Life Sci May 25, 2001;69(1):47-57.

Grant K A, Shively Calif., Nade M A, Ehrenkaufer R L, Line S W, Morton T E, Gage H D, Mach R H. Effect of social status on striatal dopamine D2 receptor binding characteristics in cynomolgus monkeys assessed with positron emission tomography. Synapse 1998 May;29(1):80-3.

Grierson J P, James M D, Pearson J R, Wilson Calif. The effect of selective D1 and D2 dopaminergic agents on sexual receptivity in the female rat. Neuropharmacology 1988 February;27(2): 181-9.

Ichikawa J, Meltzer H Y. Valproate and carbamazepine increase prefrontal dopamine release by 5-HTIA receptor activation. Eur J Pharmacol Sep. 3, 1999;380(1):R1-3.

Laakso A, Vilkman H, Kajander J, Bergman M, Haaparanta M, Solin O, Hietala J. Prediction of Detached Personality in Healthy Subjects by Low Dopamine Transporter Binding. Am J Psychiatry 200 February;157:290-292.

Leucht S, McGrath J, White P, Kissling W. Carbamazepine augmentation for schizophrenia: how good is the evidence? J Clin Psychiatry 2002 March;63(3):218-24.

Mathew S, Coplan J, Gorman J. Neurobiological Mechanisms of Social Anxiety Disorder. Am J Psychiatry 2001 October;158:1558-67.

Melis M R, Argiolas A. Dopamine and sexual behavior. Neurosci Biobehav Rev 1995 Srping;19(1):19-38.

Murakami T, Okada M, Kawata Y, Zhu G, Kamata A, Kaneko S. Determination of effects of antiepileptic drugs on SNAREs-mediated hippocampal monoamine release using in vivo microdialysis. Br J Phamacol 2001 Oct; 134(3):507-20.

Murata M, Horiuchi E, Kanazawa I. Zonisamide has beneficial effects on Parkinson's disease patients. Neurosci Res 2001 December;41(4):397-9.

Okada M, Hirano T, Mizuno K, Chiba T, Kawata Y, Kiryu K, Wada K, Tasaki H, Kaneko S. Biphasic effects of carbamazepine on the dopaminergic system in rat striatum and hippocampus. Epliepsy Res 1997 Sep;28(2):143-53.

Okada M, Kaneko S, Hirano T, Mizuno K, Kondo T, Otani K, Fukushima Y. Effects of zonisamide on dopaminergic system. Epilepsy Res 1995 November;22(3): 193-205.

Preisendorfer U, Zeise M L, Klee M R. Valproate enhances inhibitory postsynaptic potential sin hioopcampal neurons in vitro. Brain Res Dec. 1, 1987;435(1-2):213-9.

Sandoval M R, Palermo-Neto J. Effect of manipulation of the GABA system on dopamine-related behaviors. Braz J Med Biol Res 1995 January;28(1):88-99.

Schneier F R, Liebowitz M R, AbiDargham A, Zea-Ponce Y, Lin S H, Laruelle M. Low dopamine D(2) receptor binding potential in social phobia. Am J Psychiatry 2000 March; 157(3):457-9.

Southam E, Kirkby D, Higgins G A, Hagan R M. Lamotrigine inhibits monoamine uptake in vitro and modulates 5-hydroxytryptamine uptake in rats. Eur J Pharmacol Sep. 25, 1998;358(1): 19-24.

Stein M B. Neurobiological perspectives on social phobia: froma ffiliation to soology. Biol Psychiatry Dec. 15, 1998;44(12):1277-85.

Tiihonen J, Kuikka J, Bergstrom K, Lepola U, Koponen H, Leinonen E. Dopamine reuptake site densities in patients with social phobia. Am J Psychiatry 1997 February; 154(2):239-42.

Yatham L N, Liddle P F, Shiah I S, Lam R W, Ngan E, Scarrow G, Imperial M, Stoessl J, Sossi V, Ruth T J. PET study of [(18)F]6-fluoro-L-dopa uptake in neuroleptic-and mood-stabilizer-naïve first-episode nonpsychotic mania: effects of treatment with divalproex sodium.

What is claimed:

1. A process for treatment of central nervous system disorders characterized by any one or combination of the following: a) reduction in initiation and maintenance of personal attachment, social approach or interpersonal interaction; b) subjective and objective awkwardness and discomfort in interpersonal interactions; c) or oddities or peculiarities of interpersonal communication and social behavior; or d) subtle peculiarities and disorganization of social interactions insufficient to warrant a specific DSM-IV axis I diagnosis.

2. The process comprises administration of an effective amount of an anticonvulsant that possesses the property of directly, indirectly, or by downstream mechanisms modulating the activity of the neurotransmitter dopamine within the brain. Since the modulatory effect of anticonvulsants on central dopaminergic activity is not critically bound to their anticonvulsant effects, the dosages required to treat the disorders described in this document may be higher, lower or the same than those required to treat epilepsy.

3. The process for treatment according to claim 1 in which the disorder is schizoid personality disorder;

4. The process for treatment according to claim 1 in which the disorder is schizotypal personality disorder;

5. The process for treatment according to claim 1 in which the disorder is avoidant personality disorder;

6. The process for treatment according to claim 1 in which the disorder is paranoid personality disorder;

7. The process for treatment according to claim 1 in which the disorder is Aspberger's syndrome;

8. The process for treatment according to claim 1 in which the disorder is pervasive developmental disorder;

9. The process for treatment according to claim 1 in which the disorder is clinically significant social introversion that does not meet the threshold for a formal psychiatric disorder by current diagnostic standards such as DSM-IV.

10. The process for treatment according to claim 1 in which the treatment is an adequate oral dosage of valproic acid, valproate, or its enteric coated derivatives; and the daily maintenance dosage, for example is in the range of 15-60 mg/kg/d for adults and children.

11. The process for treatment according to claim 1 in which the treatment is an adequate oral dosage of carbamazepine and the daily maintenance dosage is in the range of 8-20 mg/kg/d for adults and 10-35 mg/kg/d for children.

12. The process for treatment according to claim 1 in which the treatment is an adequate oral dosage of oxcarbazepine; and the daily maintenance dosage for example is in the range of 1200-2400 mg/d for adults and 20-50 mg/kg/d for children.

13. The process for treatment according to claim 1 in which the treatment is an adequate oral dosage of zonisamide; and the usual daily maintenance dosage for example is in the range of 200-600 mg/d for adults and 4-8 mg/kg/d for children.

14. The process for treatment according to claim 1 in which the treatment is an adequate oral dosage of lamotrigine; and the daily dosage for example is in the range of 100 to 500 mg/day for adults and 1-15 mg/kg/d for children.

15. The process for treatment according to claim 1 in which the treatment is an adequate oral dosage of ethosuximide; and the daily maintenance dosage for example is in the range of 500-1500 mg/d of body weight for adults and 10-40 mg/kg/d for children.

16. The process for treatment according to claim 1 in which the treatment is an adequate oral dosage of felbamate; including for example a daily dosage in the range of 1200-3600 mg/day in adults or 15-45 mg/kg/day for children.

17. The process for treatment according to claim 1 in which the treatment is an adequate oral dosage of levetiracetam; including for example a daily maintenance dosage in the range of 1200-3600 mg/d for adults and 15-45 mg/kg/day for children.

18. The process for treatment according to claim 1 in which the treatment is an adequate oral dosage of methsuximide; including for example, a daily maintenance dosage in the range of 300-1200 mg/d for adults and 150-1200 mg/d for children.

19. The process for treatment according to claim 1 in which the treatment is an adequate oral dosage of phenytoin; and the daily maintenance dosage for example is in the range of 4-7 mg/kg for adults and 5-10 mg/kg/d for children.

20. The process for treatment according to claim 1 in which the treatment is an adequate oral dosage of primidone; and the daily maintenance dosage for example is in the range of 750-2000 mg/d for adults and 1-25 mg/kg/d for children.

21. The process for treatment according to claim 1 in which the treatment is an adequate oral dosage of tigabine, including for example, a daily maintenance dosage in the range of 32-56 mg/d for adults and 4-32 mg/d for children.

22. The process for treatment according to claim 1 in which the treatment is an adequate oral dosage of topiramate; including for example a daily maintenance dosage in the range of 200-600 mg/d for adults and 5-9 mg/kg/d for children.
